(19) United States
(12) Patent Application Publication
(10) Pub. No.: US 2026/0199649 A1
(43) Pub. Date: Jul. 16, 2026

(54) Rigid Sublingual Absorption Pouch with Manipulative Projections for Enhanced Therapeutic Delivery

(71) Applicant: Michael Scott Mortensen, Thousand Oaks, CA (US)

(72) Inventor: Michael Scott Mortensen, Thousand Oaks, CA (US)

(21) Appl. No.: 19/016,129

(22) Filed: Jan. 10, 2025

(51) Int. Cl. A61M 31/00

(52) U.S. Cl. CPC A61M 31/002; A61M 2202/0007; A61M 2202/0445; A61M 2205/0205; A61M 2205/0238; A61M 2205/581; A61M 2205/582; A61M 2205/584; A61M 2210/0643

(57) ABSTRACT

The invention discloses a rigid sublingual absorption pouch tailored to improve the delivery and efficacy of therapeutic agents, such as peptides and bioactive compounds, via the sublingual route. The pouch comprises a rigid structure housing the therapeutic agent and includes sharp or pointed manipulative projections designed to stimulate sublingual tissues, promoting increased blood flow and absorption. The device's surface texture incorporates micro-roughness to create localized abrasions or enhance mucosal permeability. Fabricated using biocompatible polymers, plant-based rigid fibers, or coated biodegradable materials, the pouch prevents premature degradation and ensures safety during use. It features a compartment that maintains the agent's integrity until manual compression or manipulation releases the agent over a controlled duration. Additional functionalities include angled projections for tissue massage, detachable components, and color-coded indicators for user guidance. This innovative design significantly improves sublingual absorption and user engagement compared to conventional delivery systems.

### COPYRIGHT NOTICE

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves all copyright rights whatsoever.

### BACKGROUND

### Field of the Invention

This invention pertains to the field of medical devices and drug delivery systems. Specifically, it focuses on sublingual delivery devices designed to optimize the absorption of therapeutic agents such as peptides and bioactive compounds through the sublingual mucosa. The invention integrates structural innovations, including a rigid pouch and user-manipulative projections, to enhance efficacy, durability, and bioavailability while maintaining safety and sustainability through biocompatible materials.

### Description of the Related Art

Sublingual drug delivery has emerged as a transformative approach for administering medications, particularly those requiring rapid onset of action or that are sensitive to degradation in the gastrointestinal (GI) tract. This method involves placing the drug under the tongue, where it is absorbed through the sublingual mucosa directly into the bloodstream. It offers significant advantages, such as bypassing first-pass metabolism and ensuring quicker systemic effects compared to oral administration. Despite its promise, the field faces challenges in delivering consistent, efficient, and user-friendly solutions, which has slowed its broader adoption.

Sublingual delivery is vital for certain classes of drugs, such as peptides, hormones, cardiovascular agents, and pain management medications, where rapid absorption and avoidance of the GI tract are critical. This method also caters to populations with swallowing difficulties, such as elderly or pediatric patients. The importance of sublingual delivery lies in its ability to deliver therapeutic agents efficiently without the invasiveness of injections. As healthcare trends move towards patient-centric and non-invasive treatment modalities, sublingual delivery systems are positioned to play a crucial role in addressing unmet medical needs and improving patient compliance.

The global sublingual drug delivery market has shown robust growth, with an estimated value of $ 3.2 billion in 2023 and a projected CAGR of 9% through 2030. The peptide therapeutics market, a primary beneficiary of sublingual delivery, is forecasted to exceed $ 40 billion by 2025 due to their growing application in chronic disease management and regenerative therapies. Studies indicate that over 60% of current sublingual systems face challenges in achieving consistent therapeutic outcomes, primarily due to passive absorption mechanisms and poor user compliance. These figures underscore the urgent need for innovation in this domain to unlock its full potential.

Several advancements have been made to improve sublingual drug delivery, targeting absorption efficiency, stability, and ease of use. Dissolvable sublingual films and tablets have gained popularity for their simplicity and convenience. Mucoadhesive polymers have been incorporated into formulations to enhance retention and contact with the mucosa. Nanotechnology-based carriers, such as nanoparticles and liposomes, have shown promise in protecting sensitive drugs and improving permeability. Microneedle patches have also been explored as a means to deliver drugs across mucosal barriers. Despite these innovations, most approaches are passive in nature, relying on the inherent properties of the sublingual mucosa without actively engaging the user or addressing mechanical stimulation as a mechanism to boost absorption.

By looking at prior art multiple advancements can be seen. For instance, a WO patent 2,015,119641A1 relates to all natural, non-toxic sublingual drug delivery systems. All natural non-toxic sublingual delivery systems improve absorption and onset profiles for numerous actives, along with bioavailability and pharmacokinetics results that are better than expected for families of moieties compounds and legacy-patented formulations.

A US patent 2,005,0155601A1 related to a sublingual drug delivery device. The drug delivery device that aerosolizes a dry powder formulation so that it forms a fine coating in the oral cavity and, more specifically, in the sublingual region of the oral cavity is described herein. In the preferred embodiment, the device contains five main parts: (i) a compressed gas canister, (ii) a dispenser body (also referred to herein as the main housing), (iii) a means for storing one or more doses of a drug formulation, (iv) a means for releasing a dose of the drug formulation such as a gas canister or spring piston and (v) a mouthpiece. Preferred configurations include circular, tubular, and rectangular. The means for storing the drug formulation may be configured to separately store one or more materials. In one embodiment, the means for storing the active agent is in the form of one or more drug discs, where the drug discs contain a plurality of blister packs, each storing one dose of the drug formulation. In another embodiment, the means for storing the active agent is a dosage cartridge containing a single dose of the drug formulation. In yet another embodiment, the drug formulation is stored on a ribbon containing a plurality of blister packs, each storing one dose of the drug formulation.

A US patent 1,02,65402B2 relates to absorption enhancers for drug administration. The present invention provides compositions and methods and for increasing the bioavailability of therapeutic agents in a subject. The compositions include at least one alkyl glycoside and at least one therapeutic agent, wherein the alkylglycoside has an alkyl chain length from about 10 to about 16 carbon atoms. In various aspects, the invention provides compositions and methods for oral delivery in the form of a tablet.

A US patent 2,01,00286100A1 relates to method and composition to improve absorption of therapeutic agents. The tablet with an enhanced dissolution profile for a medicinally active ingredient such as aspirin and methods for making the tablet. The tablet comprises a blend of crystals of the medicinally active ingredient and a dissolution aid such as sodium or calcium carbonate or bicarbonate that coats the crystals upon co-milling. The blend is then compressed to form tablets that have an enhanced dissolution profile for the medicinally active ingredient.

While current sublingual systems have advanced drug delivery, they fall short in several critical areas. Passive absorption mechanisms, as seen in dissolvable films and tablets, often result in inconsistent therapeutic outcomes due to variability in mucosal permeability among individuals. Sensitive drugs, such as peptides and bioactive compounds, remain prone to degradation before effective absorption, limiting their efficacy. User compliance is another major challenge, as these systems provide little feedback or guidance to ensure correct usage. Additionally, the lack of mechanical stimulation of the sublingual tissue results in suboptimal blood flow and reduced absorption rates. Collectively, these shortcomings highlight the need for a system that integrates active and user-interactive features.

The invention, the rigid sublingual absorption pouch with manipulative projections, is a novel solution designed to address the limitations of current sublingual drug delivery systems. The pouch is constructed with a rigid structure that securely houses the therapeutic agent, protecting it from premature degradation. Manipulative projections extending from the pouch allow users to actively stimulate sublingual tissues, enhancing blood flow and mucosal permeability. The pouch surface features a micro-rough texture to facilitate localized abrasions, further increasing the absorption rate. Made from biocompatible and saliva-resistant materials, the pouch ensures the stability of sensitive compounds until activation. Additionally, a controlled release mechanism allows for the gradual delivery of the therapeutic agent, providing consistent and effective dosing.

The invention bridges the significant gaps in the current market by introducing a user-interactive and mechanically active sublingual delivery system. Unlike passive systems, the manipulative projections engage the user, providing a physical mechanism to stimulate absorption pathways actively. This ensures more consistent and efficient absorption of sensitive compounds, such as peptides, even in individuals with lower baseline mucosal permeability. The rigid structure of the pouch prevents deformation during use, maintaining the integrity of the therapeutic agent and its controlled release profile. Additionally, ergonomic design features, such as angled projections and tactile indicators, guide users in optimal placement and manipulation, reducing improper use. By addressing the limitations of passive systems and enhancing user compliance, this invention fulfills the demand for a precise, effective, and user-friendly sublingual drug delivery solution.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

The present invention seeks to improve prior techniques and provide an advanced design and methodology for Rigid Sublingual Absorption Pouch with Manipulative Projections for Enhanced Therapeutic Delivery

It is also the objective of invention to enhance the bioavailability of therapeutic agents by utilizing a rigid sublingual pouch with active user engagement features for improved absorption.

A further objective of the present invention is to stimulate sublingual tissues using manipulative projections, increasing blood flow and tissue permeability for optimized drug delivery.

It is also an object of the invention to ensure precise and controlled release of therapeutic agents through a structural compartment that prevents dose dumping and maintains efficacy.

It is further the objective of the invention to provide a durable, rigid design that resists deformation during handling and ensures consistent performance during use.

It is also the objective of the invention to ncorporate biocompatible and environmentally friendly materials that are safe for the user and reduce environmental impact.

It is also the objective of the invention to improve user compliance through ergonomic features, such as angled projections, tactile feedback mechanisms, and flavor-enhanced formulations.

It is also the objective of the invention to prevent contamination during use by incorporating antimicrobial coatings on the pouch and manipulative projections.

It is further the objective of the invention to enable dual-functionality manipulative projections that both stimulate sublingual tissues and provide a secondary delivery mechanism when dissolvable.

The invention aims to facilitate the effective delivery of sensitive bioactive compounds, such as peptides, through a stable, saliva-resistant design.

Thus, it is the objective to accommodate combination therapies by incorporating compartmentalized designs for simultaneous or sequential release of multiple therapeutic agents.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and claims.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

This invention introduces a rigid sublingual delivery device designed to optimize the administration of therapeutic agents, such as peptides and bioactive compounds, through the sublingual route. Unlike traditional sublingual systems, which often rely solely on passive dissolution, this device incorporates active user engagement via manipulative projections and specialized surface features. The device aims to address inefficiencies in absorption, stability issues of sensitive agents, and user compliance challenges by providing a novel, durable, and interactive solution for precise and effective drug delivery.

As per its preferred embodiments, the core of the device is a rigid pouch structure designed to house the therapeutic agent securely. This rigidity ensures the pouch maintains its shape during use, preventing accidental deformation or spillage of the therapeutic agent. The pouch is fabricated from biocompatible materials such as medical-grade polymers, rigid plant-based fibers, or coated biodegradable composites. These materials are selected to ensure compatibility with sensitive bioactive compounds while resisting premature degradation in saliva. The rigid design also facilitates manual manipulation by the user, a critical aspect of the device's functionality.

As per its further embodiments, extending from at least two corners of the pouch are manipulative projections, which are integral to the device's performance. These projections are sharp or pointed and ergonomically designed to allow users to manually stimulate the sublingual tissues. This stimulation enhances blood flow and mucosal permeability, improving the absorption of the therapeutic agent. The projections also serve a secondary function as tools to guide precise placement and application of pressure under the tongue. This dual functionality sets the device apart from conventional sublingual systems by actively involving the user in the absorption process.

As per its further embodiments, surface of the pouch incorporates micro-roughness, a carefully engineered feature to enhance mucosal permeability. The textured surface interacts with the sublingual mucosa, creating localized abrasions that facilitate the penetration of the therapeutic agent. This design ensures that the absorption process is efficient without causing significant discomfort to the user. The degree of roughness can be tailored based on the therapeutic agent and intended application, balancing efficacy and user comfort.

As per its further embodiments, the pouch's material composition is critical to its functionality and safety. The device is constructed using biocompatible polymers, plant-based fibers, or biodegradable materials, ensuring that it is non-toxic and suitable for prolonged sublingual use. Coatings are applied to the pouch to provide resistance against saliva-induced degradation, maintaining the therapeutic agent's stability until activation. These materials are also environmentally friendly, aligning with sustainability goals, while ensuring compatibility with sensitive bioactive compounds.

As per its further embodiments, the structural compartment within the pouch houses the therapeutic agent, ensuring its stability until the device is manually activated. Upon manipulation, the compartment releases the agent over a predetermined duration, ensuring controlled and sustained delivery. This release mechanism is engineered to prevent dose dumping, a common issue in sublingual delivery systems, and to maximize therapeutic efficacy. The controlled release can be further optimized with features such as micro-valve systems or deformation-triggered openings, calibrated to respond to specific user-applied pressures.

As per its further embodiments, the device is particularly effective for delivering sensitive therapeutic agents, such as peptides (e.g., NAD+, BPC-157, Thymosin Beta-4), which benefit from the sublingual route due to their rapid absorption and bypass of first-pass metabolism. The structural integrity of the pouch ensures the agent remains stable until use, while the controlled release mechanism allows for gradual and effective delivery. The device's design can also accommodate combination therapies, with multiple compartments for sequential or simultaneous release of different agents. Additionally, flavor-enhanced formulations can be included to improve user compliance, especially for long-term treatments.

As per its further embodiments, device is designed to enhance user engagement and ease of use. Angled manipulative projections guide users in the correct placement and application of pressure under the tongue. Color-coded or embossed indicators on the pouch provide visual or tactile cues for optimal positioning and manipulation. Feedback mechanisms, such as a tactile “click” or audible sound, inform the user when the device is activated correctly. These user-centered features reduce the risk of improper use and ensure consistent therapeutic outcomes.

As per its further embodiments, the hygiene and safety considerations are integral to the device's design. The pouch and its manipulative projections may include antimicrobial coatings to reduce contamination risks during use. The projections can be designed to fold, retract, or dissolve after use, minimizing injury risks and providing additional functionality as dissolvable carriers for secondary therapeutic agents. The materials used are resistant to environmental factors such as heat or humidity, ensuring the device's reliability across diverse conditions. Collectively, these innovations make the rigid sublingual absorption pouch a versatile, safe, and effective solution for modern drug delivery needs.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

We claim:

1. A sublingual delivery device, comprising:
a. A rigid pouch structure configured to hold a predetermined quantity of a therapeutic agent, wherein the therapeutic agent comprises peptides or other bioactive compounds suitable for sublingual absorption;
b. Manipulative projections extending from at least two corners of the pouch, wherein said projections are sharp or pointed and designed to stimulate sublingual tissues through user manipulation;
c. A surface texture on the pouch that includes micro-roughness to enhance sublingual absorption by creating localized abrasions or increasing permeability of the sublingual mucosa;
d. A material composition selected from biocompatible polymers, plant-based rigid fibers, or coated biodegradable materials, wherein the material prevents premature degradation during use; and,
e. A structural compartment that maintains the therapeutic agent's integrity until the pouch is manually compressed or manipulated, releasing the agent over a predetermined duration.
As per claim 1, the manipulative projections are angled to mimic the function of a toothpick, allowing direct application of pressure to sublingual tissues.
As per claim 1, the pouch's rigid structure includes a feedback mechanism, such as a tactile “click,” to confirm activation of therapeutic agent release.
As per claim 1, the therapeutic agent includes peptides such as NAD+, BPC-157, or Thymosin Beta-4, formulated for sublingual bioavailability.
As per claim 1, the surface micro-roughness is designed to create localized abrasions without causing user discomfort.
As per claim 1, the material composition includes coatings that delay degradation in saliva, ensuring the therapeutic agent's stability during use.
As per claim 1, the pouch includes color-coded or embossed indicators for optimal placement under the tongue.
As per claim 1, the manipulative projections are detachable or dissolvable, allowing dual functionality for both stimulation and absorption.

2. A sublingual delivery device as claimed in claim 1, wherein the manipulative projections serve dual purposes by:
a. Stimulating sublingual tissues to enhance blood flow and absorption of the therapeutic agent; and
b. Facilitating precise application and alignment of the device under the tongue.
As per claim 2, the projections are coated with bioactive substances that dissolve during stimulation, complementing the primary therapeutic agent.
As per claim 2, the manipulative projections have antimicrobial coatings for enhanced hygiene during use.
As per claim 2, the projections fold or retract when not in use to prevent accidental injury.
As per claim 2, the projections vary in texture to provide different levels of stimulation to sublingual tissues.
As per claim 2, the device features an integrated indicator to guide users on the optimal pressure or angle for manipulation.
As per claim 2, the projections are designed to apply localized mechanical stress on sublingual vascular clusters for targeted absorption.
As per claim 2, the projections include dissolvable tips that release secondary therapeutic agents upon contact with sublingual tissues.

3. A sublingual delivery device as claimed in claim 1, wherein the rigid pouch structure is optimized for controlled release of the therapeutic agent by:
a. Incorporating a micro-valve system or a structural deformation mechanism that activates only under specific manual pressure; and
b. Ensuring a predetermined release duration to sustain therapeutic efficacy over time.
As per claim 3, the micro-valve system regulates the release of therapeutic agents in response to variations in user pressure.
As per claim 3, the rigid structure is designed to prevent accidental release during storage or handling.
As per claim 3, the pouch material is compatible with multiple therapeutic agents, enabling sequential or combination delivery.
As per claim 3, the release duration is programmable by varying the composition or structural design of the pouch.
As per claim 3, the rigid structure includes heat-resistant properties, ensuring performance in varying environmental conditions.
As per claim 3, the device features a compartmentalized structure to allow simultaneous or staged release of multiple therapeutic agents.
As per claim 3, the pouch incorporates sensory feedback mechanisms, such as a click sound or vibration, to inform the user of successful activation.
